(19) United States
(12) Patent Application Publication
(10) Pub. No.: US 2026/0295550 A1
(43) Pub. Date: Oct. 1, 2026

(54) COMPOSITION

(71) Applicant: GIVAUDAN SA, VERNIER (CH)

(72) Inventors: Emmanuel AUSSANT, Argenteuil (FR); Mathieu ZONGO, VERNIER (CH)

(21) Appl. No.: 19/483,443

(22) PCT Filed: May 8, 2024
(86) PCT No.: PCT/EP2024/062745

§ 371 Date: Nov. 11, 2025

(30) Foreign Application Priority Data
May 12, 2023 (GB) 2307053.5

(51) Int. Cl. B01J 13/04

(52) U.S. Cl. CPC B01J 13/04

(57) ABSTRACT

The present invention provides a composition comprising a water-soluble polymer matrix; and a benefit agent that is at least partially encapsulated in core-shell microcapsules comprising a core and a shell surrounding the core; wherein the composition comprises between about 54 wt % to about 85 wt % of benefit agent based on the total dry weight of the composition.

The invention also relates to a method for preparing such compositions, to a consumer product comprising such compositions and to the use of the compositions to improve the perception or enhance the performance of the benefit agent in the consumer product.

The present invention relates to a composition for controlled release of a benefit agent. In particular, the invention is concerned with a composition comprising a water-soluble matrix and a benefit agent that is at least partially encapsulated in core-shell microcapsules comprising a core and a shell surrounding the core. The invention also relates to a method of making a composition as defined herein, to a consumer product comprising the composition as defined herein and to the use of the composition and the consumer product to improve the perception or enhance the performance of the benefit agent in the consumer product.

### BACKGROUND OF THE INVENTION

It is a major challenge to develop consumer products such as household care, personal care and fabric care products which provide an optimum of exposure of receptive site to effective levels of benefit agent over a long period of time. Several studies have shown that benefit agents are perceived as being more effective if they are available at an individually tailored level at a particular time at the target site. This challenge can be addressed by utilizing encapsulated benefit agent.

It is known to incorporate encapsulated benefit agents in consumer products, such as household care, personal care, fabric care and pet care products. Benefit agents include for example fragrances, cosmetic agents, food ingredients, nutraceuticals, drugs and substrate enhancers.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, fabrics or hard household surfaces. They can also act as a means of controlling the spatio-temporal release of a benefit agent.

Spray-drying is a well-known technique for the encapsulation of benefit agents. Such spray-dried compositions are commonly prepared from an emulsion of the benefit agent to be encapsulated, which is sprayed into a drying chamber. In this process, biopolymers with surface active properties are generally used as emulsifiers which, upon spray-drying, form a water-soluble matrix in which the benefit agent becomes entrapped.

Such spray-dried compositions provide a powder format which is simple to manufacture and shows good odor benefits. Furthermore, since nowadays consumers are more aware of environmental and resource protection, those encapsulates have become even more attractive, as they are often based on bio-sourced materials. The spray-dried compositions thus have a low ecological footprint and allow for encapsulation of benefit agents with high efficiency. They also exhibit beneficial release properties.

It is important to control the release of benefit agent in a consumer product over a desired site and at a desired rate. For example, if the consumer product is a fabric care product, it is desirable to provide perception of the benefit agent, such as a fragrance, throughout the washing and rinsing cycles, at the moment the fabric is taken out of the washing machine, during drying and after the fabric has been dried.

Such a release profile is achieved by combining free, non-encapsulated benefit agent, such as a fragrance, with encapsulated benefit agent, wherein the non-encapsulated fragrance contributes essentially to enhancing fragrance perception on wet fabrics, while the encapsulated fragrance contributes essentially to enhancing fragrance perception on dry fabrics. Additionally, the encapsulated fragrance may be released during fabric handling, typically under the action of mechanical forces. Core-shell microcapsules may be used, wherein the core comprises the encapsulated fragrance and is surrounded by an impervious, frangible shell.

For example, WO2018/172514 is concerned with a composition comprising a solid carrier and a granulated powder comprising particles having a low perfume loading, which is employed as a solid scent booster. The low perfume loading was deemed necessary in order to avoid breakage during a manufacturing process requiring high shearing and for providing stability to leakage.

However, compositions employing higher benefit agent loading would provide enhanced perception of the benefit agent to the consumer.

The present invention solves the above-mentioned shortcomings.

### SUMMARY OF THE INVENTION

In a first aspect, the present invention provides a composition comprising
a) a water-soluble matrix; and
b) a benefit agent that is at least partially encapsulated in core-shell microcapsules comprising a core and a shell surrounding the core;
wherein the composition comprises between about 54 wt % to about 85 wt %, optionally about 55 wt % to about 75 wt %, preferably about 55 wt % of benefit agent based on the total dry weight of the composition.

In a further aspect, the invention provides methods of preparing the composition as described herein.

The invention further provides a consumer product comprising the composition as described herein.

The use of the composition and consumer product as described herein to improve the perception or enhance the performance of the benefit agent in the consumer product is provided in a further aspect.

### Definitions

The term “benefit agent” refers to any substance which, when added to a product, may improve the perception of this product by a consumer or may enhance the action of this product in an application. Examples of benefit agents include perfume or fragrance ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

According to the invention, “encapsulated” benefit agent refers to benefit agent that is encapsulated in a core-shell microcapsule.

By contrast, according to the invention, “non-encapsulated benefit agent refers to benefit agent that is simply entrapped (or dispersed) within the water-soluble matrix but that is not encapsulated in a core-shell microcapsule.

The term “solid” indicates that the material is in a solid state of aggregation at a temperature below about 40° C.

The term “water-soluble” indicates that the material completely dissolves in water at a temperature above about 10° C.

The term “post-rub intensity” refers to the intensity of a fragrance released upon breaking the core-shell microcapsules.

In the context of the present invention, all percentages refer to weight percentages (% w/w), unless otherwise indicated.

Dv50 or Dv(50) represents the maximum particle diameter below which 50% of the sample volume exists—also known as the median particle size by volume. It is also known as the median value of the “volume weighted distributions” or the Malvern volume weighted particle size distribution and is commonly measured using light scattering techniques.

In the context of the present invention, the term “dry weight” refers to the weight of the composition after water has been removed by spray drying.

### DETAILED DESCRIPTION

Preferred and/or optional features of the invention will now be set out. Any aspect of the invention may be combined with any other aspect of the invention unless the context demands otherwise.

Any of the preferred or optional features of any aspect may be combined, singly or in combination, with any aspect of the invention, as well as with any other preferred or optional features, unless the context demands otherwise.

The applicant has surprisingly and unexpectedly found that a composition comprising
a) a water-soluble matrix; and
b) a benefit agent that is at least partially encapsulated in core-shell microcapsules comprising a core and a shell surrounding the core;
wherein the composition comprises between about 54 wt % to about 85 wt % of benefit agent based on the total dry weight of the composition can be provided and such compositions provide improved perception of the benefit agent when employed in a consumer product compared to a composition comprising lower amounts of benefit agent.

The invention, therefore, provides a composition comprising
a) a water-soluble matrix; and
b) a benefit agent that is at least partially encapsulated in core-shell microcapsules comprising a core and a shell surrounding the core;
wherein the composition comprises between about 54 wt % to about 85 wt % of benefit agent based on the total dry weight of the composition.

### Water-Soluble Polymer Matrix

The water-soluble polymer matrix may comprise at least one material selected from the group consisting of starch, in particular water-soluble modified starch, maltodextrin, mannitol, chitosan, gum Arabic, alginate, cellulose, pectins, gelatin, polyvinyl alcohol and mixtures thereof. The resulting benefit agent encapsulates are facile and cost-effective to manufacture. Furthermore, they are prepared of naturally-based materials, which are non-toxic and biodegradable. Such encapsulates therefore have an increased consumer appeal.

When the starch is a water-soluble modified starch, such starch can be made from raw starch or pre-gelatinized starch. It can be derived from tubers, legumes, cereals and grains, for example corn starch, wheat starch, rice starch, waxy corn starch, oat starch, cassava starch, waxy barley starch, waxy rice starch, sweet rice starch, amioca starch, potato starch, tapioca starch and mixtures thereof.

The water-soluble modified starch can be selected from the group consisting of bleached starch, hydroxypropyl starch, hydroxypropyl distarch phosphate, dydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

Water-soluble modified starches have emulsifying and emulsion-stabilizing capacity. They have the ability to entrap benefit agent droplets in the form of oil-in-water emulsions due to the hydrophobic character of the starch modifying agent. The modified starches as described herein above bring numerous advantages including high emulsification and entrapment performance when the composition is dried, low viscosity, even at high solids content, and excellent oxidation resistance to ensure good fragrance and/or cosmetic preservation and stabilization of sensitive ingredients.

When the water-soluble matrix comprises a water-soluble modified starch, it can additionally comprise a material selected from the group consisting of maltodextrin, mannitol and mixtures thereof. Maltodextrin and mannitol both increase the glass transition temperature of the matrix.

Furthermore, maltodextrin is a film forming agent upon drying.

Maltodextrins are characterized by their dextrin equivalent (DE). The higher the DE, the lower is the molecular weight of the maltodextrin. In the context of the present invention, maltodextrin having different DE may be combined to provide optimized encapsulation properties. Without being bound by any theory, it is supposed that mixtures of low and high DE maltodextrins improve the packing of the water-soluble matrix in case the composition is dried, for example by spray dying.

Further to the materials stated herein above, the water-soluble matrix can additionally comprise a hemicellulose. In the context of the present invention, the expression “hemicellulose” is to be understood as a polysaccharide selected from the group consisting of glucans, in particular xyloglucans, mannans, in particular glucomannans, and xylans, in particular arabinoxylans and glucuronoxylans.

It has been found that addition of a hemicellulose to a water-soluble matrix, in particular a starch matrix, leads to a modification of the matrix after drying, improving the release properties under moisture.

The hemicellulose is preferably a xyloglucan, in particular a xyloglucan obtainable from tamarind seeds. Xyloglucans are the most abundant hemicellulose in the primary walls of non-graminaceous plants, often comprising 20 wt.-% of the dry mass of the wall. A xyloglucan has a backbone composed of 1,4-linked β-D-glucose residues. Up to 75% of the backbone residues are substituted at C6 with mono-, di-, or trisaccharide sidechains. Preferably, the hemicellulose is a xyloglucan obtainable from tamarind seeds, in particular obtained from tamarind seeds, also known as “tamarind kernel powder” or “tamarind gum”. In tamarind gum, the side chains consist of one or two α-D-xylopyranosyl units, optionally capped with β-D-galactopyranosyl, α-L-arabinofuranosyl or β-D-xylopyranosyl.

In one embodiment, the water soluble matrix is in a powder form.

Spray-drying is a well-known technique for the encapsulation of perfumes or fragrances. Such spray-dried perfume compositions are commonly prepared from an emulsion of the perfume to be encapsulated, which is sprayed into a drying chamber. In this process, biopolymers with surface active properties are generally used as emulsifiers which, upon spray-drying, form a water-soluble matrix in which the perfume becomes entrapped. Such spray-dried compositions provide a powder perfume format which is simple to manufacture and shows good odor benefits.

### Core-Shell Microcapsules

In the context of the present invention, the benefit agent is at least partially encapsulated in core-shell microcapsules comprising a core and a shell surrounding the core.

Such compositions allow for benefit agent release either through activation by mechanical action or by moisture, for instance in deodorant or antiperspirant applications. But such compositions are also particularly useful when employed as benefit agent delivery means in consumer products that require, for delivering optimal benefits, core-shell microcapsules to adhere to a substrate on which they are applied, for instance laundry detergents.

The composition of the benefit agent that is encapsulated in the core-shell microcapsules and the composition of the benefit agent that is not encapsulated in the core-shell microcapsules can be the same or different. This results in a modulated release of the same or of different benefit agent components, depending on whether the encapsulate is exposed to moisture or mechanical stresses. In particular, a sequential release of the benefit agent components may be envisioned.

In the context of the present invention, the shell of the core-shell microcapsules can comprise a polymer selected from the group consisting of a melamine-formaldehyde polymer, a urea-formaldehyde polymer, a polyurea, a polyurethane, a polyamide, a polyacrylate, a polycarbonate, and mixtures thereof.

### Thermosetting Resins

Core-shell microcapsules with a shell of a melamine-formaldehyde polymer have proven to be particularly suitable for fragrance encapsulation. They are described in the prior art, for instance in WO 2008/098387 A1, WO 2016/207180 A1, WO 2017/001672 A1 and WO 2018/197266 A1.

Suitable examples of core-shell microcapsules comprise a shell surrounding the core, wherein the shell comprises a network of cross-linked resin, wherein the resin comprises a terpolymer and a polymeric stabilizer, wherein the terpolymer comprises moieties derived from at least one polyamine; moieties derived from a milk protein or a milk protein derivative; and moieties derived from the group consisting of alkylene and alkylenoxy moieties having 1 to 6 methylene units.

Also core-shell microcapsules with a shell of a polyurea or polyurethane polymer have been successfully used for perfume encapsulation. They have the advantage to address consumer concerns with regard to residual formaldehyde in the composition. Such capsules are also described in the prior art, for instance in WO 2016/071151 A1 and WO 2019/174978 A1.

In one embodiment, the shell comprises a thermosetting resin formed by reaction of a polyfunctional amine comprising at least one amino group with at least one polyfunctional isocyanate, wherein the shell further comprises a cationic polymer comprising quaternary ammonium groups, wherein the shell further comprises a polymeric stabilizer comprising fully or partially dissociated carboxylic acid groups, such as those described in WO 2023/017014 A1.

Core-shell microcapsules with a shell of a polyacrylate, i.e. one or more monoethylenically unsaturated and/or polyethylenically unsaturated monomer(s) in polymerized form, have also been successfully used for perfume encapsulation. Such capsules are described in the prior art, for instance in WO 2013/111912 A1 or WO 2014/032920 A1.

In one embodiment, the core-shell microcapsules comprise a shell comprising a thermosetting resin formed by the reaction of shell-forming monomers comprising a polyamine and a material comprising a plurality of olefinic double bonds capable of reacting with the polyamine, such as those described in WO 2019/121738 A1.

### Polymeric Stabiliser

In one embodiment, the shell comprises a thermosetting resin formed by the reaction of shell-forming materials selected from monomers, pre-polymers and/or pre-condensates, and comprising a polymeric stabilizer that is the reaction product of a polymeric surfactant and a silane containing a functional group capable of forming covalent bonds with the shell, such as those described in WO 2019/121736 A1.

In one embodiment, the shell may comprise a polymeric stabilizer that is formed by combination of a polymeric surfactant with at least one aminosilane. The polymeric surfactant comprises a polysaccharide comprising carboxylic acid groups. The aminosilane is as defined hereinbelow. The shell may further comprise a polysaccharide, preferably a polysaccharide comprising beta (1→4) linked monosaccharide units, even more preferably a cellulose derivative, in particular selected form the group consisting of hydroxyethyl cellulose, hydroxypropylmethyl cellulose, cellulose acetate, carboxymethyl cellulose, and combinations thereof, preferably hydroxyethyl cellulose. Such capsules are described in the prior art, for instance in WO 2020/233887A1.

### Hydrated Polymer Phase and Polymeric Stabilizer

In one embodiment, the shell may comprise a hydrated polymer phase and a polymeric stabilizer at an interface between the shell and the core.

In such an arrangement, the polymeric stabilizer provides an impervious encapsulating material, whereas the hydrated polymer phase provides the desired deposition and adherence to the substrate. Furthermore, without being bound by any theory, it is surmised that the hydrated polymer phase also provides an optimal point of attack for microbial degradation.

The polymeric stabilizer may be selected from a broad range of film-forming materials and resins. Preferably, the polymeric stabilizer is highly cross-linked, in order to decrease significantly the diffusion of the encapsulated benefit agent through the shell. Preferably the imperviousness of the shell is sufficiently high to significantly prevent the leakage of the benefit agent in extractive base, such as consumer products comprising surfactants.

In one embodiment of the present invention, the polymeric stabilizer is a thermosetting resin.

Thermosetting resins are typically obtained by reacting polyfunctional monomers, such as amines, isocyanates, alcohols or phenols, chlorocarboxylic acids, (meth)acrylates, epoxides, silanes and aldehydes.

In one embodiment of the present invention, the polymeric stabilizer is formed by reaction of an aminosilane with a polyfunctional isocyanate. Such a polymeric stabilizer has the advantage of being highly crosslinked and susceptible of providing surface anchoring groups that can be used to immobilize additional materials to complete shell formation. These additional materials may comprise additional encapsulating materials, coatings and, as described in more details hereinafter, simple and complex coacervate, and hydrogels.

The aminosilane employed in the formation of the polymeric stabilizer can be selected from a compound of Formula (I).

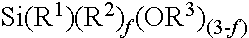

wherein R¹ is a linear or branched alkyl or alkenyl residue comprising an amine functional group; R² is each independently a linear or branched alkyl group with 1 to 4 carbon atoms; R³ is each independently a H or a linear or branched alkyl group with 1 to 4 carbon atoms; and f is 0, 1 or 2.

The silane groups may undergo polycondensation reactions with one another to form a silica network at the oil/water interface that additionally stabilizes this interface.

In one embodiment, R² and R³ are each independently methyl or ethyl.

In one embodiment, f is 0 or 1.

In one embodiment, R¹ is a C₁-C₁₂ linear or branched alkyl or alkenyl residue comprising an amine functional group. Optionally, R¹ is a C₁-C₄ linear or branched alkyl or alkenyl residue comprising an amine functional group.

In one embodiment, the amine functional group is a primary, a secondary or a tertiary amine.

In one embodiment, the at least one aminosilane is a bipodal aminosilane. By “bipodal aminosilane” it is meant a molecule comprising at least one amino group and two residues, each of these residues bearing at least one alkoxysilane moiety. Bipodal aminosilanes are particularly advantageous for forming stable oil-water interfaces, compared to conventional aminosilanes.

Without wishing to be bound by theory, it is believed that this beneficial role is due to the particular, bi-directional arrangement of the silane moieties in the molecule of a bipodal aminosilane, which allows formation of a more tightly linked silica network at the oil-water interface.

In one embodiment, the bipodal aminosilane is a compound of Formula (II).

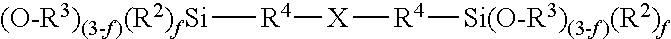

wherein X is —NR⁵—, —NR⁵—CH₂—NR⁵—, —NR⁵—CH₂—CH₂—NR⁵—, —NR⁵—CO—NR⁵—, or

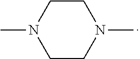

R² is each independently a linear or branched alkyl group with 1 to 4 carbon atoms;
R³ is each independently H or a linear or branched alkyl group with 1 to 4 carbon atoms;
R⁴ is each independently a linear or branched alkylene group with 1 to 6 carbon atoms;
R⁵ is each independently H, CH₃ or C₂H₅; and
f is each independently 0, 1 or 2.

In one embodiment, R² is CH₃ or C₂H₅.

In one embodiment, R³ is CH₃ or C₂H₅.

In one embodiment, R⁴ is —CH₂—, —CH₂—CH₂— or —CH₂—CH₂—CH₂—.

In one embodiment, R⁵ is H or CH₃.

In one embodiment, f is 0 or 1.

Examples of suitable bipodal aminosilanes include, but are not limited to, bis(3-(triethoxysilyl)propyl)amine, N,N′-bis(3-(trimethoxysilyl)propyl)urea, bis(3-(methyldiethoxysilyl) propyl)amine, N,N′-bis(3-(trimethoxysilyl)propyl)ethane-1,2-diamine, bis(3-(methyldimethoxysilyl)propyl)-N-methylamine, N,N′-bis(3-(triethoxysilyl) propyl)piperazine, and combinations thereof.

In one embodiment, the bipodal aminosilane is bis(3-(triethoxysilyl)propyl)amine, which has the advantage of releasing ethanol instead of more toxic and less desirable methanol during the polycondensation of the ethoxysilane groups.

The bipodal aminosilane can be a secondary aminosilane. Using a secondary bipodal aminosilane instead of a primary aminosilane decreases the reactivity of the polymeric stabilizer with respect to electrophilic species, in particular aldehydes. Hence, benefit agents containing high levels of aldehydes may be encapsulated with a lower propensity for adverse interactions between core-forming and shell-forming materials.

Other aminosilanes may also be used in combination with the aforementioned bipodal aminosilanes, in particular the aminosilanes described hereinabove.

The polyfunctional isocyanate may be selected from organic isocyanates, in which an isocyanate group is bonded to an organic residue (R—N═C═O or R—NCO). The polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule, and mixtures thereof.

Preferably, the polyfunctional isocyanate is an aromatic or an alkylaromatic isocyanate, the alkylaromatic polyfunctional isocyanate having preferably methylisocyanate groups attached to an aromatic ring. Both aromatic and methylisocyanate-substituted aromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its trifunctional nature that favors the formation of intermolecular cross-links and because of its intermediate reactivity that favors network homogeneity. This alkylaromatic polyfunctional isocyanate is commercially available under the trademark Takenate D-100 N, sold by Mitsui or under the trademark Desmodur® Quix175, sold by Covestro.

As an alternative to aromatic or alkylaromatic polyfunctional isocyanates, it may also be advantageous to add an anionically modified polyfunctional isocyanates, because of the ability of such polyfunctional isocyanates to react at the oil/water interface and even in the water phase close to the oil/water interface. A particularly suitable anionically modified polyfunctional isocyanate has Formula (III).

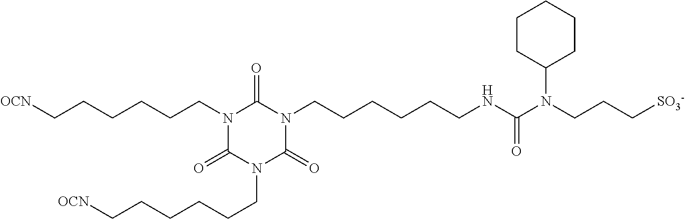

Formula (III) shows a commercially available anionically modified polyisocyanate, which is a modified isocyanurate of hexamethylene diisocyanate, sold by Covestro under the trademark Bayhydur® XP2547.

In a preferred embodiment of the present invention, polyfunctional isocyanate is 2-ethylpropane-1,2,3-triyl tris((3-(isocyanatomethyl)phenyl)carbamate). Particularly preferably, the polymeric stabilizer is formed by reaction of bis(3-(triethoxysilyl)propyl)amine and 2-ethylpropane-1,2,3-triyl tris((3-(isocyanatomethyl)phenyl)carbamate). The combination of this particular bipodal secondary aminosilane and polyfunctional isocyanate provides advantageous interface stability and release properties. The stabilized interface is sufficiently impervious to effectively encapsulate the at least one benefit agent comprised in the core and possesses the desired surface functional groups.

In one embodiment, the shells may be as described in WO 2020/207849 A1.

In preferred embodiments of the present invention the hydrated polymer phase can be a coacervate, in particular a complex coacervate.

By “complex coacervation” is meant the formation of an interfacial layer comprising a mixture of polyelectrolytes.

The phenomenon of coacervation may be observed under a light microscope, wherein it is marked by the appearance of a ring around the core composition droplet. This ring consists of the aforementioned polyelectrolyte-rich phase that has a different refractive index than the surrounding aqueous phase.

The coacervation of a polyelectrolyte is generally induced by bringing the polyelectrolyte to its isoelectric point, meaning the point where the net charge of the polyelectrolyte is zero or close to zero. This may be achieved by changing the salt concentration or the pH of the medium. In a complex coacervation, complexation occurs at the pH where one of the polyelectrolytes has an overall positive electrical charge (polycation), whereas the other polyelectrolyte has an overall negative charge (polyanion), so that the overall electrical charge of the complex is neutral.

In preferred embodiments of the present invention, the coacervate may be formed from a polycation and a polyanion.

In one embodiment, the shell can comprise a complex coacervate formed of at least one protein and at least one polysaccharide. Such core-shell capsules have proved suitable for benefit agent encapsulation and are described, for instance in WO 1996/020612 A1, WO 2001/03825 A1 or WO 2015/150370 A1.

Preferably, the pH is used as parameter driving the coacervation. Thus, the polycation preferably has a pH-dependent electrical charge. This is the case for polymers bearing primary, secondary and tertiary amino groups, such as polyamines, for example chitosan, and most proteins, for example gelatin. Proteins have the additional advantage of being prone to temperature-dependent structural transitions that may also be used to control the morphology of the coacervates. In particular, varying the temperature of some proteins may induce the formation of secondary, tertiary or quaternary structures of the protein that may also be used to control the properties of the coacervate.

Chitosan has the advantage of being derived from chitin, which is a natural polymer.

In preferred embodiments of the present invention, the polycation is selected from the group consisting of proteins, chitosan, and combinations thereof.

More particularly, the polycation can be a protein selected from the group consisting of gelatin, casein, albumin, polylysine, soy proteins, pea proteins, rice proteins, hemp proteins, potato protein, and combinations thereof.

In particularly preferred embodiments of the present invention, the at least one protein is a gelatin, even more preferably a Type B gelatin, or a potato protein.

Type B gelatin can be obtained from the alkaline treatment of collagen and is well known for its ability to form complexes with anionic polyelectrolytes, such as negatively charged polysaccharides under mild acidic conditions.

Gelatin is usually characterized by the so-called “Bloom Strength”. In the context of the present invention, the Bloom Strength refers to the rigidity of a gelatin film, as measured by so-called “Bloom Gelometer”, according to the Official Procedures of the Gelatin Manufacturers Institute of America, Inc., revised 2019, Chapter 2.1. According to this procedure, the Bloom Strength, expressed in Bloom, is equal to the weight, expressed in g, required to move vertically a standardized plunger, having a diameter of 12.5 mm, to a depth of 4 mm into a gelatin gel, which has been prepared under controlled conditions, i.e. by dissolving 6.67 wt.-% of gelatin in deionized water at 60° C., in a standardized jar, and letting the gel form for 17 hours at 10° C. The higher the weight is, the higher is the Bloom Strength of the gelatin used for making the tested gel.

In preferred embodiments of the present invention, the Type B gelatin has a Bloom Strength of 90 to 250 Bloom.

If the Bloom Strength is too low, the gel is mechanically weak and coacervates obtained therefrom may not form a self-standing layer of gelatin-rich phase around the core composition. If the Bloom Strength is too high, then the coacervates and the gelatin-rich phase obtained therefrom may be too brittle.

In preferred embodiments of the present invention, the Type B gelatin is obtainable from fish, because fish gelatin meets better acceptance within consumer than beef or pork gelatin, mainly due to health concerns, sociological context or religious rules.

Alternatively, the protein may be a vegetable protein, in particular a pea protein, a potato protein and/or a soy protein, which have the advantage of being vegan.

The polycation may be a denaturated protein. In the contrary to native proteins, denaturated proteins have been deprived from their ability to form secondary, tertiary or quaternary structures and are essentially amorphous. Such amorphous proteins may form more impervious films compared to native proteins and therefore also contribute to the encapsulating power of the shell.

Denaturation may be achieved by treating the protein with chemical or physical means, such as acid or alkaline treatment, heat or exposure to hydrogen bond disrupting agents.

In cases where the polycation is chitosan, the chitosan can have a molecular weight between 3′000 and 1′000′000 g/mol, more particularly between 10′000 and 500′000 g/mol, still more particularly between 30′000 and 300′000 g/mol.

The polyanion may be any negatively charged polymer. However, as the pH is preferably used to control coacervation, it may be more advantageous that the electrical charge of the polymer is pH-dependent. Such polymer may be selected from polymers having pendent carboxylic groups, such as methacrylic acid and acrylic acid polymers and copolymers, hydrolyzed maleic anhydride copolymers and polysaccharides bearing carboxylic groups.

In preferred embodiments of the present invention, the polyanion is a polysaccharide comprising carboxylate groups and/or sulfate groups.

Polysaccharides comprising carboxylate groups are particularly suitable for complex coacervation with proteins. This is due to the fact that the net electrical charge of these polysaccharides may be adjusted by adjusting the pH, so that the complexation with ampholytic proteins is facilitated.

Complexation occurs at the pH where the protein has an overall positive electrical charge, whereas the polysaccharide as an overall negative charge, so that the overall electrical charge of the complex is neutral. These polysaccharides include native polysaccharides, i.e. unmodified from nature, and modified polysaccharides.

The polysaccharide comprising carboxylic acid groups may comprise uronic acid units, in particular hexuronic acid units. Such polysaccharides are broadly available in nature.

The hexuronic acid units can be selected from the group consisting of galacturonic acid units, glucuronic acid units, in particular 4-O-methyl-glucuronic acid units, guluronic acid units, mannuronic acid units, and combinations thereof.

The polysaccharide comprising carboxylic acid groups may be branched. Branched polysaccharides comprising carboxylic acid groups have the advantage of forming more compact networks than linear polysaccharides and therefore may favor the imperviousness of the encapsulating shell, resulting in reduced leakage and greater encapsulation efficiency.

The carboxylate groups can be at least partially present in the form of the corresponding carboxylate salt, in particular the corresponding sodium, potassium, magnesium or calcium carboxylate salt.

In particular embodiments of the present invention, the polyanion is selected from the group consisting of pectin, gum arabic, alginate, and combinations thereof.

Among the pectins, the carboxylic acid groups can be partially present in the form of the corresponding methyl ester. The percentage of carboxylic acid groups that are present in the form of the corresponding methyl ester can be from 3% to 95%, preferably from 4% to 75%, more preferably from 5 to 50%. Pectins comprising carboxylic groups, of which 50% or more are present in the form of the corresponding methyl ester, are referred to as “high methoxylated”. Pectins comprising carboxylic acid groups, of which less than 50% are present in the form of the corresponding methyl ester, are referred to as “low methoxylated”.

Among the two variants of gum Arabic, i.e. gum acacia Senegal and gum acacia Seyal, gum acacia Senegal is preferred, owing to the higher level of glucuronic acid in gum acacia Senegal.

The hydrated polymer phase can be a hydrogel.

In context of the present invention, a “hydrogel” is a three-dimensional (3D) network of hydrophilic polymers that can swell in water, while maintaining the structure due to chemical or physical cross-linking of individual polymer chains.

Such a hydrogel can be formed by several methods at interfaces, especially by self-assembly of polyelectrolytes around existing interfaces, covalent grafting of pre-formed hydrogel particles in solution, polymerization of hydrosoluble monomers initiated at the interface and phase separation of water soluble macromolecules onto the interface.

To avoid any ambiguity, in context of the present invention, a coacervate, especially a complex coacervate, which is cross-liked, in particular by covalent bonds, is considered as a hydrogel.

The applicant has found that the use of hydrogels particularly enhances both the deposition and adherence of microcapsules on substrates, in particular on fabrics.

The hydrogel can be interlinked with the polymeric stabilizer, in particular via the functional groups present on the surface of this stabilizer.

This allows the locking of the hydrogel layer onto the polymeric stabilizer present at droplet interface, making the shell composed of a polymer composite, instead of only a blend.

Both hydrogel cross-linking and hydrogel interlinking with the polymeric stabilizer may be performed sequentially or simultaneously.

In preferred embodiments of the present invention, the hydrogel is a crosslinked coacervate, in particular a complex coacervate crosslinked with polyfunctional aldehyde, more particularly a difunctional aldehyde selected from the group consisting of succinaldehyde, glutaraldehyde, glyoxal, benzene-1,2-dialdehyde, benzene-1,3-dialdehyde, benzene-1,4-dialdehyde, piperazine-N,N-dialdehyde, 2,2′-bipyridyl-5,5′-dialdehyde, and combinations thereof. Difunctional aldehydes are known to be effective cross-linking agents for proteins.

Cross-linking of at least one protein with a first cross-linking agent followed by the addition of at least one polysaccharide to form a complex coacervate is described in WO 2021/239742 A1.

The hydrogel can be thermosensitive and possess a gelation temperature, in particular between 20° C. and 50° C., preferably between 25° C. and 40° C. When using such a hydrogel, the deposition performance of the capsules on fabric can increase, when washing the fabric at a temperature which is above hydrogel gelation temperature.

The shell can be further stabilized with a stabilizing agent. Preferably the stabilizing agent comprises at least two carboxylic acid groups. Even more preferably, the stabilizing agent is selected from the group consisting of citric acid, benzene-1,3,5-tricarboxylic acid, benzene-1,2,4-tricarboxylic acid, 2,5-furandicarboxylic acid, itaconic acid, poly(itaconic acid) and combinations thereof.

In one embodiment, the core-shell microcapsules may be as described in WO 2023/020883A1.

In one embodiment, the shell comprises a polymeric stabilizer that is formed by combination of a polymeric surfactant with at least one aminosilane; a hydrocolloid; and a linker derived from an epoxy resin. The polymeric surfactant and at least one aminosilane are as defined hereinabove. In one embodiment, hydrocolloid is selected from the group consisting of polysaccharides, such as pectin, modified starches, guar gum, locust bean gum, konjac mannan, gum arabic, gum ghatti, tragacanth, agar, alginates and carrageenan; proteins such as gelatin or potato protein; and combinations thereof. In one embodiment, the epoxy resin is selected from the group consisting of epoxidised plant oils, epoxidised alcohols, epoxidised furans, epoxidised phenols and combinations thereof.

In one embodiment, the shell comprises a first and a second polyelectrolyte which form a complex coacervate, and wherein the microcapsule comprises at least one interfacial enabler. In one embodiment, the interfacial enabler is or is derived from a diacid or a dialdehyde, such as the shells described in WO 2022/112204 A1.

In one embodiment, the shell of the microcapsules can be made of a biodegradable material or a non-biodegradable material. In one embodiment, the microcapsules are made of a biodegradable material.

In preferred embodiments of the present invention, the volume median diameter Dv(50) of the plurality of core-shell microcapsules is from 1 to 100 μm, preferably 5 to 75 μm, more preferably 8 to 60 μm, even more preferably 10 to 30 μm. Microcapsules having volume median diameter in the range from 10 to 30 μm show optimal deposition on various substrates, such as fabrics and hair.

The resultant encapsulated composition, presented in the form of a slurry of microcapsules suspended in an aqueous suspending medium, may be incorporated as such in a composition comprising a water-soluble matrix, optionally comprising benefit agent that is entrapped therein. If desired, however, the slurry may be dried to present the encapsulated composition in dry powder form. Drying of a slurry of microcapsules is conventional, and may be carried out according to techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

Combining at least two encapsulation processes has the advantage of providing different mechanisms for releasing the functional material, for example a combination of moisture-induced and mechanical stress-induced releases.

### Benefit Agent

Suitable benefit agents to be encapsulated into the core of the core-shell microcapsules of the present invention include fragrance or perfume ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

In one embodiment, the benefit agent comprises, optionally consist of at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy)butan-2-olAMBERKETAL (3,8,8,11a-tetramethyldodecahydro-1H-3,5a-epoxynaphtho[2,1-c]oxepine); AMBERMAX (2-(2,2,7,7-Tetramethyltricyclo[6.2.1.0](1,6)undec-4-en-5-yl)propan-1-ol and 2-(2,2,7,7-Tetramethyltricyclo[6.2.1.0](1,6)undec-5-en-5-yl)propan-1-ol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl)quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl) acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5(6H)-ylidene)butanal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYLACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl)pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (11H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl)methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2(3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl)acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl)propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2(3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl)pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl)propan-2-ol); TERPINEOL ALPHA (2-(4-methyl-1-cyclohex-3-enyl)propan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); Eucalyptus globulus ESS CHINA (Eucalyptus oil); Galbanum ESS (Galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In one embodiment of the present invention, more than 75%, preferably more than 80%, even more preferably more than 85%, even still more preferably more than 90%, even yet still more preferably more than 95%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl)pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl)butanal); FLORIDILE ((E)-undec-9-enenitrile); GALBANONE PURE (1-(5,5-dimethylcyclohex-1-en-1-yl)pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl)phenol); ISOJASMONE B 11 (2-hexylcyclopent-2-en-1-one); ISORALDEINE ((E)-3-methyl-4-(2,6,6-trimethylcyclohex-2-en-1-yl)but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl)phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl)propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl)propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2(3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2(3H)-one); RASPBERRY KETONE (4-(4-hydroxyphenyl)butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl)propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); Eucalyptus globulus ESS CHINA (Eucalyptus oil); Galbanum ESS (Galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil).

The above-mentioned ingredients have all been identified as not only being biodegradable, but also as being suitable for encapsulation with respect to their physical and chemical properties, such as lipophilicity, molecular size and reactivity towards shell materials. They therefore provide a useful selection of perfume ingredients for readily and reliably providing more sustainable fragrance encapsulates.

In one embodiment, the benefit agent may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

In one embodiment, the benefit agent may comprise at least one functional cosmetic ingredient.

The functional cosmetic ingredients for use in the encapsulated composition are preferably hydrophobic. Optionally, the cosmetic ingredients have a calculated octanol/water partition coefficient (C log P) of 1.5 or more, optionally 3 or more. Alternatively, the C log P of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Examples of suitable functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosemary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus spicatus kernel oil; Panthenyl triacetate; Tocopheryl acetate; Tocopherol; Naringinin; Ethyl Iinoleate; Farnesyl acetate; Farnesol; Citronellyl methyl crotonate; and Ceramide-2 (1-Stearoiyl-C18-Sphingosine, CAS-No: 100403-19-8).

In one embodiment, the benefit agent may comprise agents which suppress or reduce malodour and its perception by adsorbing odour, agents which provide a warming or cooling effect, insect repellents or UV absorbers.

In one embodiment, the benefit agent is totally encapsulated in core-shell microcapsules comprising a core and a shell surrounding the core.

If present, the non-encapsulated benefit agent can be identical or different from the encapsulated benefit agent used in the microcapsule composition as described herein above. This results in a modulated release of the same or of different odor impressions, depending on whether the encapsulate is exposed to moisture or mechanical stresses. In particular, a sequential release of the benefit agent may be envisioned.

The compositions of the present invention allow for benefit agent release either through activation by mechanical action or by moisture, for instance in deodorant or antiperspirant applications. But such compositions are also particularly useful when employed as fragrance delivery means in consumer products that require, for delivering optimal perfumery benefits, core-shell microcapsules to adhere to a substrate on which they are applied, for instance laundry detergents.

The non-encapsulated benefit agent can comprise, preferably consists of, at least one, preferably at least two, more preferably at least four, even more preferably at least eight, even still more preferably at least sixteen, biodegradable ingredient(s). The biodegradable ingredient(s) can be present at a total concentration of at least 75 wt.-%, preferably at least 80 wt.-%, more preferably at least 85 wt.-%, even more preferably at least 90 wt.-%, even still more preferably at least 95 wt.-%, relative to the total weight of the non-encapsulated benefit agent. The biodegradable ingredient(s) can be selected from the groups as defined hereinabove.

The composition of the present invention comprises between about 54 wt % to about 85 wt %, optionally about 55 wt % to about 75 wt %, preferably about 55 wt % of benefit agent based on the total dry weight of the composition. Despite prior art suggesting instability of granulated powder comprising fragrance loading greater than 30 dry wt %, the compositions of the present invention are stable in dry form.

More surprisingly, it has been found it advantageous that the composition comprises more than about 31 wt % benefit agent that is encapsulated in core-shell microcapsules with respect to the total dry weight of the composition. The olfactive performance of a composition comprising more than about 31 wt % of encapsulated benefit agent with respect to the total dry weight of the composition has been found to be higher than the olfactive performance of compositions comprising less than about 31 wt % encapsulated benefit agent.

In one embodiment, the composition comprises
a) from about 3 wt % to about 44 wt %, optionally from about 36 wt % to about 40 wt % of a water-soluble matrix based on the total dry weight of the composition;
b) from about 10 wt % to about 97 wt %, optionally between about 36.5 wt % to about 64 wt % of core-shell microcapsules comprising a core and a shell surrounding the core; and
c) from 0 to about 46 wt % non-encapsulated benefit agent, based on the total dry weight of the composition.

In one embodiment, the shell of the microcapsules comprises a thermosetting resin, in particular a shell of a melamine-formaldehyde polymer.

In one embodiment, the composition comprises:
between about 1 wt % to about 35 wt % starch sodium octenyl succinate;
between about 2 wt % to about 9 wt % mannitol;
between about 10 wt % to about 97 wt % core-shell microcapsules comprising a core and a shell surrounding the core, wherein benefit agent is encapsulated therein; and
between 0 wt % to about 46 wt % non-encapsulated benefit agent,

based on the total dry weight of the composition.

In one embodiment, the shell of the microcapsules comprises a thermosetting resin, in particular a shell of a melamine-formaldehyde polymer.

In one embodiment, the composition comprises:
between about 29 wt % to about 32 wt % starch sodium octenyl succinate;
between about 7 wt % to about 8 wt % mannitol;
between about 36.5 wt % to about 64 wt % core-shell microcapsules comprising a core and a shell surrounding the core, wherein benefit agent is encapsulated therein; and
between 0 wt % to about 26 wt % non-encapsulated benefit agent, based on the total dry weight of the composition.

In one embodiment, the shell of the microcapsules comprises a thermosetting resin, in particular a shell of a melamine-formaldehyde polymer.

In one embodiment, the composition is provided in solid form.

### Solid Carrier

In one embodiment, when the composition is in a solid form, a solid carrier is further comprised in the composition.

Solid carrier may be any particles, preferably porous particles suitable to vehicle the benefit agent on fabrics.

In one embodiment, the solid carrier is water soluble, to avoid staining of the fabrics.

Diluting the composition in a solid form with a solid carrier allows for providing formulations that are compliant with dust explosion regulations, as it is known that the explosion risk increases with the concentration of the benefit agent in a solid composition, especially when in the form of a powder.

The solid carrier may be selected from the group consisting of inorganic or organic salts or oxides of alkali metals, alkaline earth metals or transition metals, carbohydrates such as mono-, di-, and polysaccharides and derivatives thereof, polyethylene glycol (PEG), polyvinyl pyrrolidone (PVP), urea, water soluble organic solid acids, water soluble fatty alcohols or fatty acids and mixtures thereof.

In one embodiment, the solid carrier is selected from the group consisting of sodium chloride, sodium sulfate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride and combination thereof. In one embodiment, the solid carrier is a sodium salt such as sodium sulfate.

In one embodiment, the solid carrier is selected from the group consisting of mono-, di-, and polysaccharides and derivatives thereof such as sucrose, starch, cellulose, methyl cellulose, ethyl cellulose, propyl cellulose, polyols/sugar alcohols such as sorbitol, maltitol, xylitol, erythritol, and isomalt. PEG, PVP, citric acid or any water soluble solid acid, fatty alcohols or fatty acids and mixtures thereof.

In one embodiment, the solid carrier is selected from the group consisting of polyethylene glycol (PEG), polyvinyl pyrrolidone (PVP), urea, water soluble organic solid acids, water soluble fatty alcohols or fatty acids and mixtures thereof.

When a solid carrier is used, the proportion of the solid carrier can be between about 10 wt.-% to 99.9 wt.-%, preferably between about 30 wt.-% to about 97 wt.-%, even more preferably between about 50 wt.-% to about 95 wt.-%, relative to the total weight of the solid composition. Under such conditions, the solid composition may be maintained below critical explosion values even when in powder form, in terms of explosivity class and minimal ignition energy value.

As an alternative or in addition to a solid carrier, the composition according to the present invention in solid form may also comprise a flowing agent. The flowing agent may be selected from the group consisting of silicon dioxide, sodium salts, calcium salts and zeolites. Flowing agents limit the risk of powder agglomeration and clogging, and ease the transfer of the solid composition from one vessel to another.

In one embodiment, the median particle size by volume (Dv(50)) of the composition in solid form is between about 10 μm to about 10000 μm, optionally between about 25 μm to about 1000 μm, optionally between about 50 μm to about 500 μm, optionally between about 50 μm to about 250 μm.

### Methods

A further aspect of the present invention relates to a method of making a composition as described herein. The method comprises the steps of:
a) providing a slurry of microcapsules comprising encapsulated benefit agent, wherein the benefit agent is encapsulated in core-shell microcapsules comprising a core and a shell surrounding the core;
b) optionally, subjecting the slurry of microcapsules of step a) to drying, in particular spray-drying, to obtain a solid composition;
c) providing an emulsion or a suspension of a water-soluble polymer matrix material and, optionally, a non-encapsulated benefit agent;
d) optionally, subjecting the emulsion or suspension of a water-soluble polymer matrix material and, optionally, a non-encapsulated benefit agent to drying, in particular spray-drying, to obtain a solid composition;
e) blending the composition of step a) with the composition of step c) or blending the composition of step b) with the composition of step d);
f) optionally, subjecting the blend resulted from blending the composition of step a) with the composition of step c) to drying, in particular spray-drying, to obtain a solid composition;
wherein if steps b) and d) are carried out, no step f) is carried out; and
wherein the composition comprises between about 54 wt % to about 85 wt %, optionally about 55 wt % to about 75 wt %, preferably about 55 wt % of benefit agent based on the total dry weight of the composition.

The water-soluble matrix, benefit agent and core-shell microcapsules are as defined hereinabove.

In one embodiment, a solid carrier may be added during step e) if both steps b) and d) are carried out; or during step f).

The present invention also relates to a consumer product comprising a composition as described herein above.

In one embodiment, the consumer product is selected from the group consisting of a personal care product, a fabric care product, a home care product or a pet care product, preferably wherein the consumer product is a fabric care product.

In one embodiment, the consumer product is a laundry detergent.

A consumer product can contain the compositions as described herein above, preferably at a level of 0.005 to 5 wt.-%, more preferably from 0.01 to 1 wt.-%, and still more preferably from 0.02 to 0.5 wt.-%, of the consumer product.

The present invention also relates to the use of composition or the consumer product as described herein to improve the perception or enhance the performance of the benefit agent in the consumer product

The present invention is further illustrated by means of the following non-limiting examples:

### Example 1: Preparation of Compositions 1 to 4 (According to the Invention) and 5 and 6 (Comparative) by Co-Atomisation

Tap water (450 g) is weighted into a stainless steel beaker. Starch sodium octenyl succinate E1450 (in the amount according to Table 1), and mannitol 60 (in the amount according to Table 1) are subsequently weighted into the same beaker. The resulting mixture is first manually stirred with a stainless steel rod and then homogenized with an IKA T25 Ultra-Turrax Homogenizer at 13,500 rpm to obtain a homogeneous solution. To this resulting mixture, a fragrance (in the amount according to Table 1) is added. Using a two-stage high-pressure homogenizer, a stable emulsion is produced. The droplet size is controlled by dynamic light scattering to be between 1 and 5 μm.

To the emulsion is added a slurry of core-shell microcapsules (prepared according to Example 1 of WO 2016/207180 A1, in the amount according to Table 1) and agitated at 300 rpm for 10 m.

The resulting mixture is subjected to spray drying using a Mobile Niro Spray-dryer. The spray drying process parameters are as follows:
Inlet Temperature: 190° C.
Outlet Temperature: 90° C.
Peristaltic pump speed: 1.5 U/h
Fan speed: 80 kg/h

TABLE 1
Compositions 1 to 6

| | Composition | | | | | |
|---|---|---|---|---|---|---|
| Ingredient | 1 | 2 and 2′ | 3 and 3′ | 4 | 5 | 6 |
| starch sodium octenyl | 294 | 311 | 321 | 339.1 | 360 | 488 |
| succinate E1450 (g) | | | | | | |
| mannitol 60 (g) | 74 | 78 | 80 | 84.8 | 90 | 122 |
| Fragrance (g) | 0 | 138 | 220 | 412.5 | 550 | 0 |
| Microcapsule slurry | 632 | 474 | 379 | 163.7 | 0 | 361 |
| (dry weight, g) | | | | | | |
| Encapsulated | 550 | 412 | 330 | 140 | 0 | 300 |
| fragrance (g) | | | | | | |
| Shell (dry weight, g) | 82 | 62 | 49 | 23.7 | 0 | 61 |
| Total fragrance (g) | 550 | 550 | 550 | 552.5 | 550 | 300 |
| Total fragrance wt. % | 55.00 | 54.95 | 55.00 | 55.24 | 55.00 | 30.90 |
| Encapsulated fragrance | 55.00 | 41.16 | 33.00 | 14.00 | 0 | 30.90 |
| wt % | | | | | | |

### Example 2: Preparation of Compositions 2′ and 3′ (According to the Invention) by Blending

In a blender, it is added
a) a composition comprising entrapped fragrance, starch sodium octenyl succinate E1450 and mannitol in amounts according to Table 1, wherein the composition is in a powder form;
b) a composition comprising a microcapsule slurry in dry form, in the amounts according to Table 1.

The blender is closed, and the two compositions are mixed for 25 to 30 min until the resulting mixture is homogeneous.

### Example 3: Comparison of Olfactive Performance of a Laundry Detergent Comprising Compositions 1 to 6, 2′ and 3′

The olfactive performance of the compositions was assessed by a panel of 5 panelists who rated the odor intensity on a scale of 1-5 (0=no odour, 1=weak intensity, 2=acceptable intensity, 3=good intensity, 4=strong intensity and 5=very strong intensity).

For application in hand washing, the following protocol was followed:
Wash load: 2 pieces of 100% cotton terry towels. (Approximately 70 g, 30 cm×30 cm)
Water volume: 3 L
Water details: Standard tap water
Base: Powder detergent Dali unfragranced
Dosage: 0.1 wt % of spray dried composition in the detergent sample
   Wash dose: 12 g of detergent
   Weigh quantity of detergent (12 g)
   Put 3 L of water at room temperature in the sink (measured by a thermometer)
   Pour the powder in the water and dissolve the powder stirring by hands during 30 sec
   Add 2 towels in the sink one after the other
   push each towel 10 times up and down in the water
   Soak for 30 min both towels
   Recreate foam pushing each towel 10 times up and down in the water
   Prepare a basin with 3 L of water
   Rinse the 2 towels in the basin (by pushing each towel 10 times up and down in the water)
   Wring the towels by hand
   Line-dry the towels at room temperature for 24 h

The pre-rub olfactive evaluation, measuring the intensity of the perceived fragrance before rubbing, was performed after drying the hand washed towels for 24 h at room temperature (about 20° C.). The post-rub evaluation, measuring the intensity of the boost of fragrance, was performed by gently rubbing one part of the air dried towels. The performance on terry toweling of freshly prepared compositions and compositions aged for 2 weeks, at 4° C. and at 37° C., is shown in Table 2.

TABLE 2
Olfactive performace of detergents
comprising compositions 1 to 5, 2’ and 3’

| | | |
|---|---|---|
| Com- | Pre-rub | Post-rub |
| position | intensity on dry | intensity on dry |

Freshly prepared composition

| | | |
|---|---|---|
| 1 | 1.2 | 2.5 |
| 2 | 0.8 | 1.9 |
| 2’ | 1.6 | 2.3 |
| 3 | 0.5 | 1.7 |
| 3’ | 0.8 | 1.9 |
| 4 | 0.5 | 1.5 |
| 5 | 0.3 | 0 |
| 6 | 0.6 | 1.8 |

Compositions aged for 2 weeks at 4° C.

| | | |
|---|---|---|
| 1 | 1.9 | 1.9 |
| 2 | 1.5 | 1.3 |
| 2’ | 1.4 | 1.7 |
| 3 | 1.4 | 1.5 |
| 3’ | 1.7 | 1.5 |
| 4 | 1.2 | 1.3 |
| 6 | 1.1 | 1.4 |

Compositions aged for 2 weeks at 37° C.

| | | |
|---|---|---|
| 1 | 1.9 | 1.9 |
| 2 | 1.5 | 1.3 |
| 2’ | 1.4 | 1.5 |
| 3 | 1.4 | 1.4 |
| 3’ | 1.7 | 1.0 |
| 4 | 1.5 | 1.2 |
| 6 | 1.0 | 1.3 |

From the data above it can be observed that, despite the prior art teaching lack of stability for compositions with fragrance loading above 30%, the compositions of the present invention (1 to 4, 2′ and 3′) show at least as good a stability over 2 weeks (at both low and high temperature) as comparative composition 6, comprising significantly less total fragrance than the compositions of the invention, when employed in a laundry detergent. As expected, comparative composition 5 (not comprising fragrance encapsulated in core-shell microcapsules) did not show any post-rub intensity.

It can also be observed that the compositions comprising, in addition to between about 54 wt % to about 85 wt % total fragrance based on the total dry weight of the composition, at least 31 wt % fragrance encapsulated in core-shell microcapsules, perform better olfactively (compositions 1 to 3, 2′ and 3′) than the composition where the concentration of fragrance encapsulated in core-shell microcapsules is below 31 wt % (capsule 4) when employed in a laundry detergent.

### Example 4: Preparation of Compositions 7 to 10 Employing High Amounts of Fragrance

Compositions 7 to 10 employing above 54 wt %, up to about 85 wt % of total fragrance were prepared following the same method as described in Example 1, using the same ingredients as in Example 1, in the amounts according to Table 3.

TABLE 3
Compositions 7 to 10

| | Composition | | | |
|---|---|---|---|---|
| Ingredient | 7 | 8 | 9 | 10 |
| starch sodium octenyl | 1 | 1 | 2 | 3 |
| succinate E1450 (g) | | | | |
| mannitol 60 (g) | 2 | 4 | 8 | 12 |
| Fragrance (g) | 0 | 0 | 0 | 0 |
| Microcapsule slurry | 97 | 95 | 90 | 85 |
| (dry weight, g) | | | | |
| Encaps. fragrance (g) | 85 | 83.1 | 78.7 | 74.3 |
| Shell (dry weight, g) | 12 | 11.9 | 11.3 | 10.7 |
| Total fragrance (g) | 85 | 83.1 | 78.7 | 74.3 |
| Total fragrance wt. % | 85.00 | 83.10 | 78.70 | 74.30 |

Compositions 7 to 10 exhibit good stability and a stable performance over a period of time of about 12 weeks at 37° C.

1. A composition comprising
a) a water-soluble polymer matrix; and
b) a benefit agent that is at least partially encapsulated in core-shell microcapsules comprising a core and a shell surrounding the core;
wherein the composition comprises between about 54 wt % to about 85 wt % of benefit agent, based on the total dry weight of the composition.

2. The composition according to claim 1, wherein the benefit agent is totally encapsulated in core-shell microcapsules comprising a core and a shell surrounding the core.

3. The composition according to claim 1, wherein the composition comprises more than about 31 wt % benefit agent that is encapsulated in core-shell microcapsules, with respect to the total dry weight of the composition.

4. The composition according to claim 1, wherein the water-soluble polymer matrix comprises at least one material selected from the group consisting of starch, maltodextrin, mannitol, chitosan, gum Arabic, alginate, cellulose, pectins, gelatin, polyvinyl alcohol and mixtures thereof.

5. The composition according to claim 4, wherein the starch is water-soluble modified starch is selected from the group consisting of bleached starch, hydroxypropyl starch, hydroxypropyl distarch phosphate, hydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

6. The composition according to claim 5, wherein the water-soluble modified starch additionally comprises a material selected from the group consisting of maltodextrin, mannitol and mixtures thereof.

7. The composition according to claim 1, wherein the water-soluble polymer matrix additionally comprises a hemicellulose.

8. The composition according to claim 1, wherein the shell of the core-shell microcapsule comprises a melamine-formaldehyde polymer, an urea-formaldehyde polymer, a polyurea or polyurethane polymer, a polyamide, a polyacrylate, a polycarbonate, a polymeric stabilizer that is formed by combination of a polymeric surfactant with at least one aminosilane, a complex coacervate formed by cross-linking of at least one protein with a first cross-linking agent and at least one polysaccharide, or a hydrated polymer and a polymeric stabilizer formed by reaction of an aminosilane with a polyfunctional isocyanate.

9. The composition according to claim 1, wherein the benefit agent is selected from the group consisting of fragrance ingredients, cosmetic ingredients, bioactive agents, substrate enhancers, enzymes, dyes, pigments and nutraceuticals.

10. The composition according to claim 1 comprising
a) from about 3 wt % to about 44 wt % of water-soluble polymer matrix, based on the total dry weight of the composition;
b) from about 10 wt % to about 97 wt % of dry core-shell microcapsules comprising a core and a shell surrounding the core, based on the total dry weight of the composition; and
c) from 0 to about 46 wt % non-encapsulated benefit agent, based on the total dry weight of the composition.

11. The composition according to claim 1, wherein the composition is provided in solid form.

12. The composition according to claim 11 further comprising a solid carrier.

13. The compostions according to claim 11, wherein the particle size of the composition is between about 10 m to about 10000 μm.

14. A method of making a composition according to claim 1, comprising the steps of:
a) providing a slurry of microcapsules comprising encapsulated benefit agent, wherein the benefit agent is encapsulated in core-shell microcapsules comprising a core and a shell surrounding the core;
b) optionally, subjecting the slurry of microcapsules of step a) to drying to obtain a solid composition;
c) providing an emulsion or a suspension of a water-soluble polymer matrix material and, optionally, a non-encapsulated benefit agent;
d) optionally, subjecting the emulsion or suspension of a water-soluble polymer matrix material and, optionally, a non-encapsulated benefit agent to drying to obtain a solid composition;
e) blending the composition of step a) with the composition of step c) or blending the composition of step b) with the composition of step d);
f) optionally, subjecting the blend resulted from blending the composition of step a) with the composition of step c) to drying to obtain a solid composition;
wherein if steps b) and d) are carried out, no step f) is carried out; and
wherein the composition comprises between about 54 wt % to about 85 wt % of benefit agent, based on the total dry weight of the composition.

15. The method of claim 14 further comprising the step of adding a solid carrier during step e) if both steps b) and d) are carried out; or during step f).

16. A consumer product comprising the composition according to claim 1.

17. A method to improve the perception or enhance the performance of the benefit agent in the composition of claim 1 in a consumer product, wherein the method comprises adding the composition to the consumer product.

18. The composition according to claim 9, wherein the benefit agent is a fragrance ingredient.

19. The composition according to claim 12, wherein the solid carrier is water soluble.

20. The composition according to claim 19, wherein the solid carrier is selected from the group consisting of inorganic or organic salts or oxides of alkali metals, alkaline earth metals or transition metals; carbohydrates and derivatives thereof; urea; polyethylene glycol (PEG); polyvinyl pyrrolidone (PVP); water soluble organic solid acids, water soluble fatty alcohols or fatty acids and mixtures thereof.

21. The consumer product according claim 16, wherein the consumer product is selected from the group consisting of a personal care product, a fabric care product, a home care product or a pet care product.
